(19) United States
(12) Patent Application Publication
(10) Pub. No.: US 2022/0015995 A1
(43) Pub. Date: Jan. 20, 2022

(54) AEROSOL FOAM DEVICE CONTAINING A COMPOSITION RICH IN FATTY SUBSTANCES

(71) Applicant: L'Oreal, Paris (FR)

(72) Inventors: Lionel Aubert, Saint-Ouen (FR); Raquel Stefani, Saint-Ouen (FR)

(73) Assignee: L'Oreal, Paris (FR)

(21) Appl. No.: 17/311,288

(22) PCT Filed: Dec. 19, 2019
(86) PCT No.: PCT/EP2019/086339

§ 371 Date: Jun. 4, 2021

(30) Foreign Application Priority Data
Dec. 20, 2018 (FR) FR1873700

(51) Int. Cl. A61K 8/04; A61Q 5/06; A61Q 5/12; A61K 8/31; A61K 8/36; A61K 8/44; A61K 8/37

(52) U.S. Cl. CPC A61K 8/046; A61Q 5/06; A61Q 5/12; A61K 2800/596; A61K 8/361; A61K 8/442; A61K 8/375; A61K 8/31

(57) ABSTRACT

The invention relates to an aerosol device containing a cosmetic composition comprising at least 30% by weight of liquid fatty substances, particular surfactants and water. The invention also relates to a process for cosmetically treating keratin fibres, in particular human keratin fibres such as the hair, comprising the application to said keratin fibres of said cosmetic composition. The invention also relates to the use of said cosmetic composition for conditioning and/or shaping keratin fibres, in particular human keratin fibres such as the hair.

The invention relates to an aerosol foam device containing a cosmetic composition comprising at least 30% by weight of liquid fatty substances, particular surfactants and water.

The invention also relates to a cosmetic process for treating keratin fibres, in particular human keratin fibres such as the hair, comprising the application to said keratin fibres of said cosmetic composition dispensed from said aerosol device.

The invention also relates to the use of said cosmetic composition dispensed from said aerosol device for conditioning and/or shaping keratin fibres, in particular human keratin fibres such as the hair.

Cosmetic oils are usually used for caring for and/or shaping keratin fibres, in particular human keratin fibres such as the hair. These cosmetic oils are usually presented in serum, emulsion or spray form. However, the cosmetic and working properties of these products, such as the application and spreading qualities, are not optimal. Furthermore, these products are not easy to rinse off the hands after application.

In order to improve the working properties of these cosmetic oils, it is known practice to present them in foam form. However, it is difficult to form a foam from a composition comprising a large amount of oily compound(s). As a result, the foams presented as “cosmetic oil” on the market contain a substantial and predominant aqueous phase.

Thus, there is a real need to provide a composition in foam form comprising a large amount of oil for conditioning and/or shaping keratin fibres, in particular human keratin fibres such as the hair, which does not have the drawbacks mentioned above, i.e. a composition which has good foam properties and which is easy to apply to keratin fibres.

The composition must also spread easily on the keratin fibres, while at the same time affording them good cosmetic and sensory properties.

It must also have good rinseability from the user's hands and from the keratin fibres.

It has been discovered, surprisingly, that a combination, in an aerosol device, of at least one liquid fatty substance in a high concentration of at least 30% by weight, of at least one amphoteric surfactant, of at least one nonionic surfactant, of water and of at least one propellant gas makes it possible to achieve the objectives presented above.

Specifically, the aerosol device containing this particular combination makes it possible to form a foam comprising a high content of oil that is easy to measure out, which has good properties, notably in terms of texture, in particular in terms of hardness, volume and homogeneity, and also good application and spreading properties on keratin fibres.

The composition thus dispensed also makes it possible to give keratin fibres good cosmetic properties, notably in terms of sheen, disentangling, softness, lightness, suppleness and smoothness. It also makes it possible to improve their shaping and their manageability, to reduce frizziness, to control their volume and to improve the curl definition.

The composition also has good rinseability and is easy to remove from the user's hands and/or from the keratin fibres after use.

One subject of the invention is thus an aerosol device containing a cosmetic composition comprising:
one or more fatty substances with a melting point of less than or equal to 25° C., in a content of greater than or equal to 30% by weight, relative to the total weight of the composition,
one or more amphoteric surfactants,
one or more nonionic surfactants,
water, and
one or more propellants.

A subject of the invention is also a cosmetic process for treating keratin fibres, in particular human keratin fibres such as the hair, comprising a step of applying to said keratin fibres a composition dispensed from an aerosol device as defined previously.

A subject of the invention is also the use of a composition dispensed from an aerosol device as defined previously, for conditioning and/or shaping keratin fibres.

Other subjects, characteristics, aspects and advantages of the invention will emerge even more clearly on reading the description and the examples that follow.

In the text hereinbelow, unless otherwise indicated, the limits of a range of values are included in that range, notably in the expressions “between” and “ranging from . . . to . . . ”.

The expression “at least one” used in the present description is equivalent to the expression “one or more”.

Liquid Fatty Substances

The cosmetic composition contained in the aerosol device according to the present invention comprises one or more fatty substances with a melting point of less than or equal to 25° C., preferably less than or equal to 20° C., at atmospheric pressure (1.013×10⁵ Pa), in a content of greater than or equal to 30% by weight, relative to the total weight of the composition.

According to the invention, this or these fatty substances are also referred to as “liquid fatty substance(s)” or “oil(s)”.

For the purposes of the present invention, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name MDSC 2920 by the company TA Instruments. In the present patent application, the melting points are determined at atmospheric pressure (1.013×10⁵ Pa).

The term “fatty substance” means an organic compound that is insoluble in water at 25° C. and at atmospheric pressure (1.013×10⁵ Pa) (solubility of less than 5% by weight, preferably less than 1% by weight and even more preferentially less than 0.1% by weight). They bear in their structure at least one hydrocarbon-based chain including at least 6 carbon atoms and/or a sequence of at least two siloxane groups. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

Advantageously, the liquid fatty substances that may be used in the present invention are not (poly)oxyalkylenated, that is to say that they do not comprise a [O—CₙH₂ₙ]ₘ group in which n is an integer ranging from 2 to 6 and m is an integer equal or greater than 1, and do not contain any —COOH functions.

The term “non-silicone fatty substance” refers to a fatty substance not containing any Si—O bonds and the term “silicone fatty substance” refers to a fatty substance containing at least one Si—O bond.

More particularly, the liquid fatty substance(s) with a melting point of less than or equal to 25° C. according to the invention are chosen from C₆ to C₁₆ liquid hydrocarbons, liquid hydrocarbons comprising more than 16 carbon atoms, non-silicone oils of animal origin, oils of triglyceride type of plant or synthetic origin, fluoro oils, liquid fatty alcohols, liquid fatty acid and/or fatty alcohol esters other than triglycerides, and silicone oils, and mixtures thereof.

It is recalled that the fatty alcohols, esters and acids more particularly contain at least one saturated or unsaturated, linear or branched hydrocarbon-based group, comprising from 6 to 30 and better still from 8 to 30 carbon atoms, which is optionally substituted, in particular, with one or more hydroxyl groups (in particular 1 to 4). If they are unsaturated, these compounds may comprise one to three conjugated or unconjugated carbon-carbon double bonds.

As regards the C₆ to C₁₆ liquid hydrocarbons, they are linear, branched or optionally cyclic, and are preferably alkanes. Examples that may be mentioned include hexane, cyclohexane, undecane, dodecane, isododecane, heptane, octane, nonane, tridecane or isoparaffins, such as isohexadecane or isodecane, and mixtures thereof.

The liquid hydrocarbons comprising more than 16 carbon atoms may be linear or branched, of mineral or synthetic origin, and are preferably chosen from liquid paraffin or liquid petroleum jelly, polydecenes, possibly hydrogenated polyisobutene such as Parleam®, and mixtures thereof.

A hydrocarbon-based oil of animal origin that may be mentioned is perhydrosqualene.

The triglyceride oils of plant or synthetic origin are preferably chosen from liquid fatty acid triglycerides including from 6 to 30 carbon atoms, for instance heptanoic or octanoic acid triglycerides, or alternatively, for example, sunflower oil, corn oil, soybean oil, pumpkin oil, grapeseed oil, sesame oil, hazelnut oil, apricot oil, macadamia oil, arara oil, sunflower oil, castor oil, avocado oil, caprylic/capric acid triglycerides, for instance those sold by the company Stéarinerie Dubois or those sold under the names Miglyol® 810, 812 and 818 by the company Dynamit Nobel, jojoba oil and shea butter oil, and mixtures thereof.

As regards the fluoro oils, they may be chosen from perfluoromethylcyclopentane and perfluoro-1,3-dimethylcyclohexane, sold under the names Flutec® PCl and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-trifluoromethyl perfluoromorpholine sold under the name PF 5052® by the company 3M.

The liquid fatty alcohols that are suitable for use in the invention are more particularly chosen from linear or branched, saturated or unsaturated alcohols, preferably unsaturated or branched alcohols, including from 6 to 30 carbon atoms and preferably from 8 to 30 carbon atoms. Examples that may be mentioned include octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol and linoleyl alcohol, and mixtures thereof.

As regards the liquid esters of fatty acids and/or of fatty alcohols other than the triglycerides mentioned above, mention may be made notably of esters of saturated or unsaturated, linear C₁ to C₂₆ or branched C₃ to C₂₆ aliphatic monoacids or polyacids and of saturated or unsaturated, linear C₁ to C₂₆ or branched C₃ to C₂₆ aliphatic monoalcohols or polyalcohols, the total carbon number of the esters being greater than or equal to 6 and more advantageously greater than or equal to 10.

Preferably, for the esters of monoalcohols, at least one from among the alcohol and the acid from which the esters of the invention are derived is branched.

Among the monoesters, mention may be made of dihydroabietyl behenate; octyldodecyl behenate; isocetyl behenate; isostearyl lactate; lauryl lactate; linoleyl lactate; oleyl lactate; isostearyl octanoate; isocetyl octanoate; octyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononate; octyldodecyl erucate; oleyl erucate; ethyl palmitate and isopropyl palmitate, such as 2-ethylhexyl palmitate, 2-octyldecyl palmitate; alkyl myristates such as isopropyl 2-octyldodecyl myristate; isobutyl stearate; 2-hexyldecyl laurate, and mixtures thereof.

Preferably, among the monoesters of monoacids and of monoalcohols, use will be made of ethyl palmitate and isopropyl palmitate, alkyl myristates such as isopropyl myristate or ethyl myristate, isocetyl stearate, 2-ethylhexyl isononanoate, isodecyl neopentanoate and isostearyl neopentanoate, and mixtures thereof.

Still within the context of this variant, esters of C₄ to C₂₂ dicarboxylic or tricarboxylic acids and of C₁ to C₂₂ alcohols and esters of mono-, di- or tricarboxylic acids and of C₂ to C₂₆ di-, tri-, tetra- or pentahydroxy alcohols may also be used.

Mention may notably be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates, and mixtures thereof.

The composition may also comprise, as fatty ester, sugar esters and diesters of C₆ to C₃₀ and preferably C₁₂ to C₂₂ fatty acids. It is recalled that the term “sugar” refers to oxygen-bearing hydrocarbon-based compounds bearing several alcohol functions, with or without aldehyde or ketone functions, and which include at least 4 carbon atoms. These sugars may be monosaccharides, oligosaccharides or polysaccharides.

Examples of suitable sugars that may be mentioned include sucrose, glucose, galactose, ribose, fucose, maltose, fructose, mannose, arabinose, xylose and lactose, and derivatives thereof, notably alkyl derivatives, such as methyl derivatives, for instance methylglucose.

The sugar esters of fatty acids may be chosen notably from the group comprising the esters or mixtures of esters of sugars described previously and of linear or branched, saturated or unsaturated C₆ to C₃₀ and preferably C₁₂ to C₂₂ fatty acids. If they are unsaturated, these compounds may comprise one to three conjugated or unconjugated carbon-carbon double bonds.

The esters according to this variant may also be chosen from mono-, di-, tri- and tetraesters, polyesters, and mixtures thereof.

These esters may be, for example, oleates, laurates, palmitates, myristates, behenates, cocoates, stearates, linoleates, linolenates, caprates, arachidonates or mixtures thereof such as, in particular, the mixed oleo-palmitate, oleo-stearate and palmito-stearate esters.

More particularly, use is made of monoesters and diesters and in particular sucrose, glucose or methylglucose mono- or dioleates, stearates, behenates, oleopalmitates, linoleates, linolenates and oleostearates, and mixtures thereof.

Mention may be made, by way of example, of the product sold under the name Glucate® DO by the company Amerchol, which is a methylglucose dioleate.

Preferably, use will be made of a liquid ester of a monoacid and of a monoalcohol.

The silicone oils that may be used in the composition according to the present invention may be volatile or non-volatile, cyclic, linear or branched silicone oils, which are unmodified or modified with organic groups, and preferably have a viscosity, at 25° C. and atmospheric pressure (1.013×10⁵ Pa), from 5×10⁶ to 2.5 m²/s at 25° C., and preferably 1×10⁵ to 1 m²/s.

Preferably, the silicone oils are chosen from polydialkylsiloxanes, notably polydimethylsiloxanes (PDMS), and liquid polyorganosiloxanes including at least one aryl group.

These silicone oils may also be organomodified. The organomodified silicone oils that may be used in accordance with the invention are preferably liquid silicones as defined above and including in their structure one or more organofunctional groups attached via a hydrocarbon-based group, chosen, for example, from amine groups and alkoxy groups.

Organopolysiloxanes are defined in greater detail in Walter Noll's Chemistry and Technology of Silicones (1968), Academic Press. They may be volatile or non-volatile.

When they are volatile, the silicone oils are more particularly chosen from those with a boiling point of between 60° C. and 260° C., and even more particularly from:

(i) cyclic polydialkylsiloxanes including from 3 to 7 and preferably from 4 to 5 silicon atoms. These are, for example, octamethylcyclotetrasiloxane sold notably under the name Volatile Silicone® 7207 by Union Carbide or Silbione® 70045 V2 by Rhodia, decamethylcyclopentasiloxane sold under the name Volatile Silicone® 7158 by Union Carbide, and Silbione® 70045 V5 by Rhodia, and mixtures thereof.

Mention may also be made of cyclocopolymers of the dimethylsiloxane/methylalkylsiloxane type, such as Volatile Silicone® FZ 3109 sold by the company Union Carbide.

Mention may also be made of mixtures of cyclic polydialkylsiloxanes with organosilicon compounds, such as the mixture of octamethylcyclotetrasiloxane and tetra(trimethylsilyl)pentaerythritol (50/50) and the mixture of octamethylcyclotetrasiloxane and oxy-1,1′-bis(2,2,2′,2′,3,3′-hexatrimethylsilyloxy)neopentane;

(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pages 27-32—Todd & Byers Volatile Silicone Fluids for Cosmetics.

Non-volatile polydialkylsiloxanes are preferably used.

These silicone oils are more particularly chosen from polydialkylsiloxanes, among which mention may be made mainly of polydimethylsiloxanes bearing trimethylsilyl end groups. The viscosity of the silicones is measured at 25° C. according to ASTM standard 445 Appendix C.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s;
the Viscasil® oils from General Electric and certain oils of the SF series (SF 96, SF 18) from General Electric.

Mention may also be made of polydimethylsiloxanes bearing dimethylsilanol end groups, known under the name dimethiconol (CTFA), such as the oils of the 48 series from the company Rhodia.

The organomodified silicones that may be used in accordance with the invention are silicones as defined above and including in their structure one or more organofunctional groups attached via a hydrocarbon-based group.

As regards the liquid polyorganosiloxanes including at least one aryl group, they may notably be polydiphenylsiloxanes, and polyalkylarylsiloxanes functionalized with the organofunctional groups mentioned previously.

The polyalkylarylsiloxanes are particularly chosen from linear and/or branched polydimethyl/methylphenylsiloxanes and polydimethyl/diphenylsiloxanes with a viscosity ranging from 1×10⁵ to 5×10⁻² m²/s at 25° C.

Among these polyalkylarylsiloxanes, examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

Among the organomodified silicones, mention may be made of polyorganosiloxanes including:
substituted or unsubstituted amino groups, such as the products sold under the names GP 4 Silicone Fluid and GP 7100 by the company Genesee or the products sold under the names Q2 8220 and Dow Corning 929 or 939 by the company Dow Corning. The substituted amino groups are in particular C1 to C4 aminoalkyl groups;
alkoxy groups,
hydroxyl groups.

Preferably, the fatty substance(s) with a melting point of less than or equal to 25° C. are chosen from liquid C₆ to C₁₆ hydrocarbons and mixtures thereof and also polydialkylsiloxanes, notably polydimethylsiloxanes (PDMS), and in particular cyclic polydialkylsiloxanes, such as cyclomethicones, and more particularly cyclopentasiloxane. Even more preferentially, said fatty substance(s) are chosen from liquid C₆ to C₁₆ hydrocarbons and mixtures thereof, such as isododecane.

The content of the fatty substance(s) with a melting point of less than or equal to 25° C. is greater than or equal to 30% by weight, preferably greater than or equal to 50% by weight and more preferentially ranges from 60% to 90% by weight, relative to the total weight of the composition.

Amphoteric Surfactants

The cosmetic composition contained in the aerosol device according to the invention also comprises one or more amphoteric surfactants.

The amphoteric surfactants that may be used in the invention may be optionally quaternized secondary or tertiary aliphatic amine derivatives, in which the aliphatic group is a linear or branched chain including from 8 to 22 carbon atoms, said amine derivatives containing at least one anionic group, for instance a carboxylate, sulfonate, sulfate, phosphate or phosphonate group.

Mention may in particular be made of (C₅-C₂₀)alkylbetaines, sulfobetaines, (C₅-C₂₀)alkylsulfobetaines, (C₅-C₂₀)alkylamido(C₁-C₆)alkylbetaines, such as cocoamidopropylbetaine, (C₅-C₂₀)alkylamido(C₁-C₆)alkylsulfobetaines, and also mixtures thereof.

Among the derivatives of optionally quaternized secondary or tertiary aliphatic amines that may be used, mention may also be made of the products having the respective structures (A1) and (A2) below:

Rₐ—CON(Z)CH₂—(CH₂)ₘ—N⁺(R_{b})(R_{c})(CH₂COO⁻) (A1)

in which:
Rₐ represents a C₁₀-C₃₀ alkyl or alkenyl group derived from an acid Rₐ—COOH preferably present in hydrolysed coconut kernel oil, or a heptyl, nonyl or undecyl group,
R_{b} represents a β-hydroxyethyl group,
R_{c} represents a carboxymethyl group;
m is equal to 0, 1 or 2, and
Z represents a hydrogen atom or a hydroxyethyl or carboxymethyl group;

R_{a′}—CON(Z)CH₂—(CH₂)ₘ—N(B)(B′) (A2)

in which:
B represents —CH₂CH₂OX′, with X′ representing —CH₂—COOH, CH₂—COOZ′, —CH₂CH₂—COOH, —CH₂CH₂—COOZ′, or a hydrogen atom,
B′ represents —(CH₂)_{z}—Y′, with z=1 or 2, and Y′ representing —COOH, —COOZ′, —CH₂—CHOH—SO₃H or CH₂—CHOH—SO₃Z′,
m′ is equal to 0, 1 or 2,
Z represents a hydrogen atom or a hydroxyethyl or carboxymethyl group,
Z′ represents an ion derived from an alkali metal or alkaline-earth metal, such as sodium, potassium or magnesium; an ammonium ion; or an ion derived from an organic amine and notably from an amino alcohol, such as monoethanolamine, diethanolamine or triethanolamine, monoisopropanolamine, diisopropanolamine or triisopropanolamine, 2-amino-2-methyl-1-propanol, 2-amino-2-methyl-1,3-propanediol and tris(hydroxymethyl)aminomethane, and
R_{a′} represents a C₁₀-C₃₀ alkyl or alkenyl group of an acid R_{a′}COOH preferably present in hydrolysed linseed oil or in coconut kernel oil, an alkyl group, notably a C₁₇ alkyl group, and its iso form, or an unsaturated C₁₇ group.

The compounds corresponding to formula (A2) are particularly preferred.

Among the compounds of formula (A2) for which X′ represents a hydrogen atom, mention may be made of the compounds known under the (CTFA) names sodium cocoamphoacetate, sodium lauroamphoacetate, sodium caproamphoacetate and sodium capryloamphoacetate.

Other compounds of formula (A2) are known under the (CTFA) names disodium cocoamphodiacetate, disodium lauroamphodiacetate, disodium caproamphodiacetate, disodium capryloamphodiacetate, disodium cocoamphodipropionate, disodium lauroamphodipropionate, disodium caproamphodipropionate, disodium capryloamphodipropionate, lauroamphodipropionic acid and cocoamphodipropionic acid.

As examples of compounds of formula (A2), mention may be made of the cocoamphodiacetate sold by the company Rhodia under the trade name Miranol® C₂M Concentrated, the sodium cocoamphoacetate sold under the trade name Miranol Ultra C 32 and the product sold by the company Chimex under the trade name Chimexane HA.

Use may also be made of the compounds of formula (A3):

R_{a″}—NH—CH(Y″)—(CH₂)ₙ—C(O)—NH—(CH₂)_{n′}—N(Rₐ)(Rₑ) (A3)

in which:
R_{a″} represents a C₁₀-C₃₀ alkyl or alkenyl group of an acid R_{a″}—C(O)OH, which is preferably present in hydrolysed linseed oil or in coconut kernel oil;
Y″ represents the group —C(O)OH, —C(O)OZ″, —CH₂—CH(OH)—SO₃H or the group CH₂—CH(OH)—SO₃—Z″, with Z″ representing a cation resulting from an alkali metal or alkaline-earth metal, such as sodium, an ammonium ion or an ion resulting from an organic amine;
R_{d} and Rₑ, independently of each other, represent a C₁-C₄ alkyl or hydroxyalkyl radical; and
n and n′, independently of each other, denote an integer ranging from 1 to 3.

Among the compounds of formula (A3), mention may in particular be made of the compound classified in the CTFA dictionary under the name sodium diethylaminopropyl cocoaspartamide and notably the compound sold by the company Chimex under the name Chimexane HB.

The amphoteric surfactant(s) are advantageously chosen from (C₈-C₂₀)alkylbetaines, (C₅-C₂₀)alkylamido(C₃-C₈)alkylbetaines, and mixtures thereof, and preferably from cocoylbetaine and cocamidopropylbetaine, and mixtures thereof.

The content of the amphoteric surfactant(s) advantageously ranges from 0.1% to 10% by weight, preferably from 0.25% to 5% by weight, more preferentially from 0.5% to 2% by weight, relative to the total weight of the composition.

Nonionic Surfactants

The cosmetic composition contained in the aerosol device according to the invention also comprises one or more nonionic surfactants.

The nonionic surfactants that may be used are described, for example, in the Handbook of Surfactants by M. R. Porter, published by Blackie & Son (Glasgow and London), 1991, pages 116-178.

Examples of nonionic surfactants that may be mentioned include the following nonionic surfactants:
oxyalkylenated (C₅-C₂₄)alkylphenols;
saturated or unsaturated, linear or branched, oxyalkylenated or glycerolated C₈ to C₄₀ alcohols, including one or two fatty chains;
saturated or unsaturated, linear or branched, oxyalkylenated C₈ to C₃₀ fatty acid amides;
esters of saturated or unsaturated, linear or branched, C₈-C₃₀ acids and of polyols or polyethylene glycols;
preferably oxyethylenated esters of saturated or unsaturated, linear or branched, C₈ to C₃₀ acids and of sorbitol;
esters of fatty acids and of sucrose;
(C₅-C₃₀)alkyl(poly)glucosides, (C₅-C₃₀)alkenyl(poly)glucosides, which are optionally oxyalkylenated (0 to 10 oxyalkylene units) and comprise from 1 to 15 glucose units, (C₅-C₃₀)alkyl(poly)glucoside esters;
saturated or unsaturated oxyethylenated plant oils;
condensates of ethylene oxide and/or of propylene oxide;
N—(C₅-C₃₀)alkylglucamine and N—(C₅-C₃₀)acylmethylglucamine derivatives;
aldobionamides;
amine oxides;
oxyethylenated and/or oxypropylenated silicones;
and mixtures thereof.

The oxyalkylene units are more particularly oxyethylene or oxypropylene units, or a combination thereof, preferably oxyethylene units.

The number of moles of ethylene oxide and/or of propylene oxide preferably ranges from 1 to 250, more particularly from 2 to 100 and better still from 2 to 50; the number of moles of glycerol notably ranges from 1 to 50 and better still from 1 to 10.

Advantageously, the nonionic surfactants according to the invention do not comprise any oxypropylene units.

As examples of glycerolated nonionic surfactants, use is preferably made of monoglycerolated or polyglycerolated C₈ to C₄₀ alcohols, comprising from 1 to 50 mol of glycerol and preferably from 1 to 10 mol of glycerol.

Among the glycerolated alcohols, it is more particularly preferred to use the C₈/C₁₀ alcohol containing 1 mol of glycerol, the C₁₀/C₁₂ alcohol containing 1 mol of glycerol and the C₁₂ alcohol containing 1.5 mol of glycerol.

The nonionic surfactant(s) that may be used in the composition according to the invention are preferentially chosen from:
oxyethylenated C₈ to C₄₀ alcohols comprising from 1 to 100 mol of ethylene oxide, preferably from 2 to 50 and more particularly from 2 to 40 mol of ethylene oxide and including one or two fatty chains;
saturated or unsaturated oxyethylenated plant oils comprising from 1 to 100 and preferably from 2 to 50 mol of ethylene oxide;
(C₅-C₃₀)alkyl(poly)glucosides, which are optionally oxyalkylenated (0 to 10 EO) and comprising 1 to 15 glucose units;
monoglycerolated or polyglycerolated C₈ to C₄₀ alcohols, comprising from 1 to 50 mol of glycerol and preferably from 1 to 10 mol of glycerol;
saturated or unsaturated, linear or branched, oxyalkylenated C₈ to C₃₀ fatty acid amides;
esters of saturated or unsaturated, linear or branched, C₈-C₃₀ acids and of polyols or polyethylene glycols;
and mixtures thereof.

The nonionic surfactant(s) that may be used are preferably chosen from esters of saturated or unsaturated, linear or branched C₈ to C₃₀ acids and of polyols, and mixtures thereof, preferably esters of saturated or unsaturated, linear or branched C₈ to C₃₀, preferably C₈ to C₂₂, preferentially C₁₀ to C₁₈ and better still C₁₀ to C₁₄ acids and of glycerol, and mixtures thereof.

Advantageously, the content of the nonionic surfactant(s) ranges from 0.01% to 10% by weight, preferably from 0.05% to 5% by weight, preferentially from 0.1% to 2% by weight, relative to the total weight of the composition.

Propellants

The aerosol device according to the invention also comprises one or more propellants.

According to the invention, the propellant(s) are present in the cosmetic composition in the aerosol device, which may be either a monoblock device or a bag system of bag-on-valve type.

Advantageously, the total content of the propellant(s) ranges from 0.1% to 40% by weight, preferably from 0.1% to 30% by weight, relative to the total weight of the composition.

According to a first embodiment of the invention, the propellant(s) that may be used in the aerosol device of the present invention are advantageously chosen from liquefied gases and mixtures thereof, preferably dimethyl ether, chlorinated and/or fluorinated hydrocarbons, such as trichlorofluoromethane, dichlorodifluoromethane, chlorodifluoromethane, 1,1,1,2-tetrafluoroethane, chloropentafluoroethane, 1-chloro-1,1-difluoroethane, 1,1-difluoroethane, 1,3,3,3-tetrafluoropropene; volatile hydrocarbons, notably such as C3-5 alkanes, for instance propane, isopropane, n-butane, isobutane, pentane, and mixtures thereof, more preferentially from propane, isopropane, n-butane, isobutane, and mixtures thereof.

According to the first embodiment, the content of the liquefied gas(es) preferably ranges from 1% to 30% by weight, more preferentially from 5% to 20% by weight, relative to the total weight of the composition.

According to a second embodiment of the invention, the propellant(s) that may be used in the aerosol device of the present invention are advantageously chosen from compressed gases and mixtures thereof, preferably from air, nitrogen, carbon dioxide, and mixtures thereof; more preferentially, the propellant is carbon dioxide.

According to this second embodiment, the content of the compressed gas(es) preferably ranges from 0.1% to 10% by weight, more preferentially from 0.5% to 5% by weight, relative to the total weight of the composition.

The cosmetic composition contained in the aerosol device according to the invention also comprises water.

The water content is advantageously greater than or equal to 5% by weight; preferably, the water content ranges from 5% to 30% by weight, more preferentially from 10% to 20% by weight, relative to the total weight of the composition.

Additives

The cosmetic composition contained in the aerosol device according to the present invention may also optionally comprise one or more additives, different from the compounds of the invention and among which mention may be made of cationic or anionic surfactants, and mixtures thereof, fatty substances other than the liquid fatty substances of the invention, cationic, anionic, nonionic or amphoteric polymers, or mixtures thereof, antidandruff agents, anti-seborrhoea agents, vitamins and provitamins including panthenol, sunscreens, mineral or organic pigments, sequestrants, plasticizers, solubilizers, acidifying agents, mineral or organic thickeners, notably polymeric thickeners, opacifiers or nacreous agents, antioxidants, hydroxy acids, fragrances, preserving agents, pigments and fillers.

Needless to say, a person skilled in the art will take care to select this or these optional additional compounds such that the advantageous properties intrinsically associated with the composition according to the invention are not, or are not substantially, adversely affected by the envisaged addition(s).

The above additives may generally be present in an amount, for each of them, of between 0 and 20% by weight relative to the total weight of the composition.

Aerosol Device

The aerosol device comprising the cosmetic composition according to the present invention is constituted of a container containing said composition and of a means for dispensing said composition.

According to the invention, the aerosol device may either be a monoblock device or a bag system of the bag-on-valve type.

Preferably, the aerosol device according to the invention makes it possible to dispense said composition in foam form.

The composition according to the invention is advantageously packaged under pressure, in an aerosol device, for example a monobloc device, which comprises a dispensing means and a container.

The dispensing means is generally constituted of a valve and a diffuser via which the composition of the invention is delivered, preferably in foam form.

The container containing the pressurized composition may be opaque or transparent. It may be made of glass, polymer material or metal, and may optionally be coated with a protective varnish coat.

A subject of the invention is also a cosmetic process for treating keratin fibres, in particular human keratin fibres such as the hair, comprising a step of applying to said keratin fibres a composition dispensed from an aerosol device as defined previously.

The composition according to the invention may be applied to dry or wet keratin materials that have optionally been washed with shampoo.

A subject of the invention is also the use of a composition dispensed from an aerosol device according to the present invention, for conditioning and/or shaping keratin fibres, in particular human keratin fibres such as the hair.

The examples that follow serve to illustrate the invention without, however, being limiting in nature.

### EXAMPLES

In the examples that follow, all the amounts are indicated as weight percentages of starting material relative to the total weight of the composition.

### Example 1 (Propellant: Liquefied Gas)

Composition A according to the present invention was prepared using the ingredients whose contents are indicated in the table below (amounts expressed in g).

TABLE 1

| | Composition A |
|---|---|
| Glucose | 2.96 |
| Refined coconut kernel oil | 0.70 |
| Cocamidopropylbetaine at | 2.62 |
| 30% in aqueous solution | |
| Isododecane | 61.57 |
| Glyceryl laurate | 0.15 |
| Dimethiconol | 5.13 |
| Glycerol | 0.05 |
| Fragrance, preserving agents | qs |
| Isobutane/propane/butane mixture | 13 |
| Water | qs 100 |

The mixture is packaged in an aerosol device constituted of an aluminium container with a volume of 150 ml, equipped with a press valve (nozzle 0.61 mm without internal restriction and without additional gas intake) and a foam diffuser (Mars from Precision).

The foam generated is creamy, very airy, and spreads easily on the hair.

The hair thus treated is easy to disentangle and has a soft feel, both when wet and after drying. The hair is manageable.

### Example 2 (Propellant: Compressed Gas)

Composition B according to the present invention was prepared using the ingredients whose contents are indicated in the table below.

TABLE 2

| | Composition B |
|---|---|
| Glucose | 3.34 |
| Refined coconut kernel oil | 0.79 |
| Cocamidopropylbetaine at | 2.56 |
| 30% in aqueous solution | |
| Isododecane | 69.57 |
| Glyceryl laurate (90/10 mono/di) | 0.17 |
| Dimethiconol | 5.8 |
| Glycerol | 0.06 |
| Fragrance, preserving agents | qs |
| Carbon dioxide | 1.7 |
| Water | qs 100 |

The composition is packaged in an aerosol device constituted of an aluminium container with a volume of 150 ml, equipped with a press valve (nozzle 0.61 mm without internal restriction and without additional gas intake) and a foam diffuser (Mars from Precision).

The foam generated is rich, creamy and glossy, and spreads easily on the hair.

The hair thus treated is easy to disentangle and has a soft feel, both when wet and after drying. The hair is manageable.

### Example 3

Compositions C and D according to the present invention were prepared using the ingredients whose contents are indicated in the tables below.

TABLE 3

| | Composition C |
|---|---|
| Isopropyl palmitate | 33.0 |
| Isopropyl myristate | 19.8 |
| Cocamidopropylbetaine at | 0.71% AM* |
| 30% in aqueous solution | |
| Caprylic/capric triglyceride | 19.8 |
| Glycerol laurate | 0.15 |
| Glycerin | 0.05 |
| Fragrance, preserving agents | qs |
| Isobutane/propane/butane mixture | 10 |
| Water | qs 100 |

TABLE 4

| | Composition D |
|---|---|
| Cyclopentasiloxane and | 13.5 |
| Dimethiconol (85,3/14,7)** | |
| Cyclopentasiloxane | 59.0 |
| Cocamidopropylbetaine at | 0.71% AM* |
| 30% in aqueous solution | |
| Caprylic/capric triglyceride | 19.8 |
| Glycerol laurate | 0.15 |
| Glycerin | 0.05 |
| Fragrance, preserving agents | qs |
| Isobutane/propane/butane mixture | 10 |
| Water | qs 100 |

*AM: active material
**Mirasil C-DML from Bluestar

1. Aerosol device containing a cosmetic composition comprising:
one or more fatty substances with a melting point of less than or equal to 25° C., in a content of greater than or equal to 30% by weight, relative to the total weight of the composition,
one or more amphoteric surfactants,
one or more nonionic surfactants,
water, and
one or more propellants.

2. Aerosol device according to claim 1, characterized in that the fatty substance(s) with a melting point of less than or equal to 25° C. are chosen from liquid C₆ to C₁₆ hydrocarbons, liquid hydrocarbons comprising more than 16 carbon atoms, non-silicone oils of animal origin, oils of triglyceride type of plant or synthetic origin, fluoro oils, liquid fatty alcohols, liquid esters of fatty acid and/or of fatty alcohol other than triglycerides, silicone oils, and mixtures thereof, and preferably from liquid C₆ to C₁₆ hydrocarbons, cyclic polydialkylsiloxanes, and mixtures thereof, more preferentially chosen from liquid C₆ to C₁₆ hydrocarbons, and mixtures thereof.

3. Aerosol device according to claim 1, characterized in that the content of the fatty substance(s) with a melting point of less than or equal to 25° C. is greater than or equal to 30% by weight, preferably greater than or equal to 50% by weight and more preferentially ranges from 60% to 90% by weight, relative to the total weight of the composition.

4. Aerosol device according to claim 1, characterized in that the amphoteric surfactant(s) are chosen from (C₈-C₂₀)alkylbetaines, (C₅-C₂₀)alkylamido(C₃-C8)alkylbetaines, and mixtures thereof, and preferably from cocoylbetaine and cocamidopropylbetaine, and mixtures thereof.

5. Aerosol device according to claim 1, characterized in that the content of the amphoteric surfactant(s) ranges from 0.1% to 10% by weight, preferably from 0.25% to 5% by weight and more preferentially from 0.5% to 2% by weight, relative to the total weight of the composition.

6. Aerosol device according to claim 1, characterized in that the nonionic surfactant(s) are chosen from oxyalkylenated (C₅-C₂₄)alkylphenols; saturated or unsaturated, linear or branched, oxyalkylenated or glycerolated C₈ to C₄₀ alcohols, including one or two fatty chains; saturated or unsaturated, linear or branched, oxyalkylenated C₈ to C₃₀ fatty acid amides; esters of saturated or unsaturated, linear or branched C₈ to C₃₀ acids and of polyols or of polyethylene glycols; esters, which are preferably oxyethylenated, of saturated or unsaturated, linear or branched C₈ to C₃₀ acids and of sorbitol; esters of fatty acids and of sucrose; (C₅-C₃₀)alkyl(poly)glucosides, (C₈-C₃₀)alkenyl(poly)glucosides, which are optionally oxyalkylenated (0 to 10 oxyalkylene units) and comprising from 1 to 15 glucose units, (C₅-C₃₀)alkyl (poly)glucoside esters; oxyethylenated, saturated or unsaturated plant oils; condensates of ethylene oxide and/or of propylene oxide; N—(C₅-C₃₀)alkylglucamine and N—(C₅-C₃₀)acylmethylglucamine derivatives; aldobionamides; amine oxides; oxyethylenated and/or oxypropylenated silicones; and mixtures thereof.

7. Aerosol device according to claim 1, characterized in that the nonionic surfactant(s) are chosen from esters of saturated or unsaturated, linear or branched C₈ to C₃₀ acids and of polyols, and mixtures thereof, preferably from esters of saturated or unsaturated, linear or branched C₈ to C₃₀, preferably C₈ to C₂₂, preferentially C₁₀ to C₁₈ and better still C₁₀ to C₁₄ acids and of glycerol, and mixtures thereof.

8. Aerosol device according to claim 1, characterized in that the content of the nonionic surfactant(s) ranges from 0.01% to 10% by weight, preferably from 0.05% to 5% by weight and more preferentially from 0.1% to 2% by weight, relative to the total weight of the composition.

9. Aerosol device according to claim 1, characterized in that the water content is greater than or equal to 5% by weight; preferably, the water content ranges from 5% to 30% by weight and better still from 10% to 20% by weight, relative to the total weight of the composition.

10. Aerosol device according to claim 1, characterized in that the propellant(s) are chosen from liquefied gases, preferably from dimethyl ether, chlorinated and/or fluorinated hydrocarbons, volatile hydrocarbons, and mixtures thereof, more preferentially from propane, isopropyl, n-butane, isobutane, and mixtures thereof.

11. Aerosol device according to claim 10, characterized in that the content of the liquefied gas(es) ranges from 1% to 30% by weight and better still from 5% to 20% by weight relative to the total weight of the composition.

12. Aerosol device according to claim 1, characterized in that the propellant(s) are chosen from compressed gases, and mixtures thereof, preferably from air, nitrogen, carbon dioxide, and mixtures thereof, more preferentially carbon dioxide.

13. Aerosol device according to claim 12, characterized in that the content of the compressed gas(es) ranges from 0.1% to 10% by weight and better still from 0.5% to 5% by weight relative to the total weight of the composition.

14. Cosmetic process for treating keratin fibres, in particular human keratin fibres such as the hair, comprising a step of applying to said keratin fibres a composition dispensed from an aerosol device as defined in claim 1.

15. Use of a composition dispensed from an aerosol device according to claim 1, for conditioning and/or shaping keratin fibres.
